(19) United States
(12) Patent Application Publication
(10) Pub. No.: US 2010/0322888 A1
(43) Pub. Date: Dec. 23, 2010

(54) Emulsion Composition for Hair Treating Agents

(75) Inventors: Akimasa Mochizuki, Sayama-shi (JP); Hirokatsu Okazaki, Sayama-shi (JP)

Correspondence Address: THE WEBB LAW FIRM, P.C., 700 KOPPERS BUILDING, 436 SEVENTH AVENUE, PITTSBURGH, PA, 15219, US

(73) Assignee: ARIMINO CO., LTD., Tokyo (JP)

(21) Appl. No.: 12/526,116

(22) PCT Filed: Dec. 5, 2007
(86) PCT No.: PCT/JP2007/073488

§ 371 Date: Aug. 6, 2009

(30) Foreign Application Priority Data
Feb. 8, 2007 (JP) 2007-029789

(51) Int. Cl. A61K 8/86; A61K 8/91; A61Q 5/06; A61Q 5/04

(52) U.S. Cl. 424/70.2; 424/70.11; 424/70.16; 424/70.6

(57) ABSTRACT

Hair treating emulsion compositions are resistant to drying after applied to hair and have a formulation permitting easy application to hair. The emulsion composition includes a lubricant, a surfactant and water and further includes 0.5 to 15 wt % of at least one polyhydric alcohol selected from the group consisting of glycerol, diglycerol and dipropylene glycol; 0.005 to 1 wt % of a highly polymerized polyethylene glycol; and 0.05 to 10 wt % of lanolin and/or a lanolin derivative.

### FIELD OF THE INVENTION

The present invention relates to emulsion compositions for hair treating agents. In more detail, the invention relates to hair treating emulsion compositions which contain a lubricant, a surfactant and water and specific amounts of a specific polyhydric alcohol, a highly polymerized polyethylene glycol and lanolin and/or lanolin derivative and which are suitable as hair coloring agents or hair perming agents.

### BACKGROUND OF THE INVENTION

It has been popular to color or wave hair with hair treating agents such as hair coloring agents and hair perming agents. However, the hair treating agents often damage hair and cause hair breakage, split ends, dry hair or lackluster hair. There has recently been an increasing awareness that such damages to hair should be minimized.

Hair coloring agents or hair perming agents, after applied to hair, are allowed to stand for a prescribed time to let the hair treating agents penetrate into the hair sufficiently. After the hair is allowed to stand, it is brushed with a comb held substantially perpendicular to the scalp and thereby the hair treating agent is spread homogeneously. This so-called combing through treatment permits the hair treating agents to adhere to hair more homogenously and increases the effects of the agents.

In the event that the hair treating agent has dried to some degree during the standing for a prescribed time, the dried agent portions block the comb when the hair is combed through. Forcibly combing through the hair results in more serious damages to the treated hair.

Patent Document 1 discloses hair coloring agent compositions whereby damages to the hair are suppressed and hair can be dyed while keeping smooth touch and luster. In detail, the hair coloring agent compositions essentially contain (A) one or two kinds of keratin protein hydrolyzates and keratin protein hydrolyzate derivatives, (B) a polyhydric alcohol, (C) an amphoteric polymer, (D) a cationic polymer and (E) a nonionic polymer. The hair coloring agent compositions achieve a certain level of improvements in touch and luster of colored hair and coloring effects. However, the hair coloring agent compositions do not have sufficient resistance to drying after applied to hair.

Further, depending on the formulations, the hair treating agents applied to hair tend to stay disproportionately near the scalp or cannot be spread adequately to the tip of hair.

Accordingly, there has been a need for hair treating agents having a formulation such that the agents are easily applied uniformly.

### Patent Document 1: JP-A-2002-97121

### SUMMARY OF THE INVENTION

If hair treating agents show sufficient resistance to drying after applied to hair, hair can be combed through without catching the comb and damages to treated hair can be reduced. Such agents preferably have an appropriate formulation permitting easy application to hair.

It is therefore an object of the invention to provide hair treating emulsion compositions which show resistance to drying after applied to hair and have a formulation permitting easy application to hair.

An emulsion composition for hair treating agents according to the present invention comprises a lubricant, a surfactant and water and further comprises:

0.5 to 15 wt % of at least one polyhydric alcohol selected from the group consisting of glycerol, diglycerol and dipropylene glycol;

0.005 to 1 wt % of a highly polymerized polyethylene glycol; and

0.05 to 10 wt % of lanolin and/or a lanolin derivative.

The emulsion composition for hair treating agents preferably further comprises 0.05 to 3 wt % of an amphoteric polymer, and the amphoteric polymer is preferably a dimethyldiallylammonium chloride/acrylamide/acrylic acid copolymer.

The highly polymerized polyethylene glycol preferably has a viscosity average molecular weight in the range of 20,000 to 4,500,000. The lanolin and/or lanolin derivative is preferably a lanolin fatty acid octyldodecyl.

The emulsion composition for hair treating agents may be suitably used as a hair coloring agent or a hair perming agent.

According to the invention, the specific polyhydric alcohol, highly polymerized polyethylene glycol and lanolin and/or lanolin derivative are blended in the specified amounts, whereby synergetic effects are achieved between anti-drying effects by glycerol and other components, and good finish brought about by the highly polymerized polyethylene glycol and anti-drying effects by the lanolin and the like. The emulsion compositions for hair treating agents consequently show appropriate viscous properties and excellent drying resistance.

### ADVANTAGEOUS EFFECTS OF THE INVENTION

The hair treating emulsion compositions used as hair treating agents such as hair coloring agents or hair perming agents are sufficiently resistant to drying when the agents applied to hair are allowed to stand for a prescribed time, and the hair can be combed through without catching a comb. As a result, the emulsion compositions reliably prevent damages to treated hair in contrast to the existing hair treating agents.

The hair treating emulsion compositions have a formulation with appropriate viscous properties and spreadability. The compositions are easily applied to hair uniformly, and have good rinsability and finger-combing properties in hair rinsing.

The emulsion compositions give smoothness and luster to the treated hair.

The hair treating emulsion compositions having these effects are suitably used as hair coloring agents or hair perming agents.

### PREFERRED EMBODIMENTS OF THE INVENTION

The emulsion compositions for hair treating agents will be described in detail hereinbelow.

The hair treating emulsion compositions of the invention contain a lubricant, a surfactant and water and further contain specific components in specified amounts. The lubricants used herein are components generally used in hair treating agents, referring to lubricants other than lanolin and/or lanolin derivatives described later. Exemplary lubricants include higher alcohols such as lauryl alcohol, myristyl alcohol, cetyl alcohol, stearyl alcohol, cetostearyl alcohol, behenyl alcohol, isostearyl alcohol, oleyl alcohol, 2-hexyldecanol, 2-octyldodecanol and 2-decyltetradecanol; oils and fats such as glycerides including avocado oil, jojoba oil, macadamia nut oil and olive oil; hydrocarbons such as liquid paraffin, solid paraffin, isoparaffin and squalane; esters such as isopropyl myristate and octyldodecyl myristate; and silicone derivatives such as dimethylpolysiloxane, methylphenylpolysiloxane, polyether-modified silicones and amino-modified silicones.

The surfactants used herein are surfactants generally used in hair treating agents, such as anionic surfactants, cationic surfactants, amphoteric surfactants and nonionic surfactants. They mainly work to increase dispersibility of the components contained in the hair treating emulsion compositions. Examples of the surfactants include cetostearyltrimethylammonium, polyoxyethylene lauryl ether sulfate, laurylhydroxysulfobetaine and polyoxyethylene cetyl ether.

The water used herein is purified water such as ion exchanged water or distilled water. The water mainly works to facilitate dissolution or dispersion of the components contained in the hair treating emulsion compositions.

The emulsion compositions for hair treating agents according to the present invention contain the above-described lubricants, surfactants and water, and further contain specific amounts of at least one polyhydric alcohol selected from the group consisting of glycerol, diglycerol and dipropylene glycol, a highly polymerized polyethylene glycol, and lanolin and/or a lanolin derivative.

These specific components and the hair treating emulsion compositions of the invention will be described in detail hereinbelow.

### <Polyhydric Alcohols>

The hair treating emulsion compositions contain at least one polyhydric alcohol selected from the group consisting of glycerol, diglycerol and dipropylene glycol. These specific polyhydric alcohols prevent the drying of the emulsion compositions, so that the compositions used as hair coloring agents or hair perming agents can be applied to hair and be combed through without dried portions of the composition catching the comb.

In general, hair coloring agents or hair perming agents applied to desired hair portions should be allowed to stand as such for approximately several minutes to several tens of minutes to let the agents penetrate the hair sufficiently. During this period, water evaporates from the agent and the agent is gradually dried. The hair is subsequently combed through to apply the agent uniformly from the root to the tip of hair, to promote the penetration of the agent into the hair, or to bring out the effects of the agent at a higher level. Combing through the hair is a treatment which spreads the hair treating agent on the hair with a comb. Thus, if the agent has dried to an advanced stage, the dried portions of the agent catch the comb to make smooth combing difficult. Consequently, the combing places a load on hair and can be a major cause of damaged hair.

The hair treating emulsion compositions of the invention contain the specific polyhydric alcohols capable of sufficiently preventing the drying of the hair treating agents. Because the agents are prevented from drying, there will be no dried portions of the agents which can catch a comb when the hair is combed through, thereby reducing damages to the treated hair. Furthermore, the specific polyhydric alcohols give appropriate viscous properties to the hair treating emulsion compositions. Thus, the emulsion compositions have a cream formulation that is resistant to sagging, has excellent spreadability and is easily applied to hair. A single or two or more kinds of the specific polyhydric alcohols may be used.

The hair treating emulsion compositions generally contain the specific polyhydric alcohols at 0.5 to 15 wt %, preferably 1.5 to 12 wt %, and more preferably 3 to 10 wt % based on 100 wt % of the hair treating emulsion composition. The specific polyhydric alcohols used in this amount effectively provide drying resistance and give appropriate viscous properties in combination with the highly polymerized polyethylene glycol described later, permitting easy application of the hair treating emulsion compositions to hair. When two or more kinds of the polyhydric alcohols are used, the total amount thereof falls in the above range.

### <Highly Polymerized Polyethylene Glycols>

The hair treating emulsion compositions of the invention contain a highly polymerized polyethylene glycol. The highly polymerized polyethylene glycols are nonionic ethylene oxide polymers having a viscosity average molecular weight of not less than 20,000. Specific examples include PEG-2M, PEG-5M, PEG-7M, PEG-9M, PEG-14M, PEG-20M, PEG-23M, PEG-45M, PEG-90M and PEG-115M. Highly polymerized polyethylene glycols having a viscosity average molecular weight of 20,000 to 4,500,000 are desirable. In detail, PEG-2M, PEG-5M, PEG-7M, PEG-9M, PEG-14M, PEG-20M, PEG-23M, PEG-45M and PEG-90M are preferable, and PEG-9M is more preferable. The highly polymerized polyethylene glycols may be used singly, or two or more kinds may be used in combination.

The highly polymerized polyethylene glycols give viscous properties to the hair treating emulsion compositions and, in combination with the specific polyhydric alcohols, provide a cream formulation that is resistant to sagging, has excellent spreadability and is easily applied to hair. The highly polymerized polyethylene glycols work to give a good finish and good touch to hair that is colored or permed with the hair treating agent. Further, the use of the highly polymerized polyethylene glycols improves the easiness to rinse off the agent, namely, rinsability. Accordingly, the highly polymerized polyethylene glycols ensure that appropriate rinsability is not deteriorated even by adding very greasy lubricants such as lanoline and lanolin derivatives described later.

The hair treating emulsion compositions generally contain the highly polymerized polyethylene glycols at 0.005 to 1 wt %, preferably 0.015 to 0.5 wt %, and more preferably 0.025 to 0.3 wt % based on 100 wt % of the hair treating emulsion composition. The highly polymerized polyethylene glycols used in this amount, in combination with the specific polyhydric alcohols, provide appropriate viscous properties without any potential to inhibit the effects of lanolin and lanolin derivatives described later, and give a good finish and good touch to the treated hair as well as high rinsability. When two or more kinds of the highly polymerized polyethylene glycols are used, the total amount thereof falls in the above range.

### <Amphoteric Polymers>

Amphoteric polymers work together with the highly polymerized polyethylene glycols to accentuate the effects of the highly polymerized polyethylene glycols. In more detail, the amphoteric polymers give appropriate viscous properties to the hair treating emulsion compositions and give a good finish and good touch to hair that is colored or permed with the hair treating composition.

Examples of the amphoteric polymers include N-methacryloyl ethyl N,N-dimethylammonium α-N-methylcarboxybetaine/butyl methacrylate copolymer (product name: Yukaformer AM-75, manufactured by Mitsubishi Chemical Corporation), hydroxypropyl acrylate/butylaminoethyl methacrylate/octylamide acrylate copolymer (product name: Amphomer 28-4910, manufactured by National Starch), dimethyldiallylammonium chloride/acrylic acid copolymer (product name: Marcoat 280 and 295, manufactured by Calgon), dimethyldiallylammonium chloride/acrylamide/acrylic acid copolymer (product name: Marcoat Plus 3330 (polymer purity: 50 wt %), manufactured by Calgon; Marcoat Plus 3331, manufactured by Calgon) and acrylic acid/methyl acrylate/methacrylamidopropyltrimethylammonium chloride copolymer (product name: Marcoat 2001, manufactured by Calgon). In particular, dimethyldiallylammonium chloride/acrylamide/acrylic acid copolymer is preferable.

The hair treating emulsion compositions generally contain the amphoteric polymers at 0.05 to 3 wt %, preferably 0.1 to 2 wt %, and more preferably 0.2 to 1.5 wt % based on 100 wt % of the hair treating emulsion composition.

### <Lanolin and Lanolin Derivatives>

The hair treating emulsion compositions contain lanolin and/or lanolin derivative (hereinafter, collectively the lanolin(s)). The lanolins further enhance the drying resistance effects brought about by the specific polyhydric alcohols and highly polymerized polyethylene glycols in combination. As a result, the hair treating emulsion compositions applied to hair can be rinsed with good finger-combing properties and give a good touch to the treated hair.

The lanolins can be hydrated by, for example, forming a hydrogen bonding with water. In the production of the hair treating emulsion compositions, they work to take in sufficient water into the compositions in the stirring step and thereby accentuate the drying resistance effects brought about by the specific polyhydric alcohols and highly polymerized polyethylene glycols.

The lanolins used in the invention include lanolin, lanolin fatty acid PEG ester, lanolin fatty acid diethanolamide, lanolin fatty acid triethanolamine ester, lanolin fatty acid octyldodecyl, lanolin fatty acid isopropyl, polyoxyethylene lanolin (30), polyoxyethylene lanolin (50), polyoxyethylene lanolin (75), polyoxyethylene lanolin alcohol and sulfonated lanolin. Of these lanolins, lanolin, lanolin fatty acid octyldodecyl and lanolin fatty acid isopropyl are preferred, and lanolin fatty acid octyldodecyl is more preferred. The lanolins may be used singly, or two or more kinds may be used in combination.

The hair treating emulsion compositions generally contain the lanolins at 0.05 to 10 wt %, preferably 0.1 to 7 wt %, and more preferably 0.25 to 5 wt % based on 100 wt % of the hair treating emulsion composition. The lanolins used in this amount enhance the drying resistance effects brought about by the specific polyhydric alcohols and highly polymerized polyethylene glycols in combination, while the stickiness inherent to the lanolins is appropriately suppressed. As a result, the hair treating compositions applied as hair coloring agents or hair perming agents can be rinsed with good finger-combing properties. Further, the lanolins supply oil to hair while their stickiness is appropriately suppressed in the compositions. Thus, luster and smoothness as well as good finish are given to the hair treated. When two or more kinds of the lanolins are used, the total amount thereof falls in the above range.

The hair treating emulsion compositions contain the specific components in the specified amounts, whereby the components work together in harmony without deteriorating the effects of the respective components and achieve still enhanced effects.

### <Other Components>

The hair treating emulsion compositions of the invention may contain other components as required while still achieving the object of the invention. Such other components include moisturizers such as sorbitol, hyaluronic acid and pyrrolidonecarboxylates; hydrolyzates of proteins such as collagen, keratin, soy protein and wheat protein, and derivatives of such hydrolyzates; chelating agents such as edetates; stabilizers such as phenacetin; plant extracts; vitamins; and pigments.

When the emulsion compositions for hair treating agents are used as hair coloring agents or hair perming agents, for example, components as described below are used together.

### <Hair Coloring Agents>

When the emulsion compositions are used as hair coloring agents, dyes such as paraphenylenediamine, resorcin and meta-aminophenol, alkaline agents such as ammonia water, and oxidizers such as hydrogen peroxide may be used together with the compositions. Further, known cosmetic components may be appropriately used, with examples including salts such as ammonium bicarbonate and ammonium chloride, perfumes, preservatives, UV absorbents, antioxidants such as sodium sulfite, fungicides and moisturizers.

A hair coloring agent generally contains a first agent and a second agent, and these agents are mixed together when used for hair treatment. Generally the first agent and the second agent are mixed at 1:1 (weight ratio), but the ratio is not limited thereto in the invention as long as the mixture of the agents corresponds to the emulsion composition of the invention. That is, the components of the emulsion composition of the invention may be contained in either of the first and the second agent as long as the amounts thereof in the hair coloring agent obtained by the mixing are as described hereinabove relative to 100 wt % of the hair coloring agent.

### <Hair Perming Agents>

When the hair treating emulsion compositions of the invention are used as hair perming agents, reducing agents such as thioglycolic acid, alkaline agents such as ammonia water, and oxidizers such as sodium bromate may be used together with the compositions.

Further, known cosmetic components may be appropriately used, with examples including perfumes, preservatives, UV absorbents, antioxidants such as sodium sulfite, fungicides and moisturizers.

A hair perming agent generally contains a first agent as a reducing agent and a second agent as an oxidizer, and hair is given the first agent first and then the second agent. Generally these agents are used at 1:1 (weight ratio), but the ratio is not limited thereto in the invention as long as either of the first agent and the second agent is the emulsion composition of the invention. Both the first agent and the second agent may correspond to the emulsion compositions according to the invention. That is, the components of the emulsion composition of the invention should be contained in the above-described amounts at least in 100 wt % of the first agent or 100 wt % of the second agent. When both the first and the second agent correspond to the hair treating compositions of the invention, they produce synergetic effects and provide superior results.

### <Emulsion Compositions for Hair Treating Agents>

The emulsion compositions for hair treating agents according to the invention may be prepared by appropriately stirring, mixing, heating, cooling, dissolving or dispersing (emulsifying) the above-described components by known methods. They have a cream formulation and appropriate viscous properties. Therefore, the hair treating agents containing the emulsion compositions are cream-like agents with balanced viscous properties that can be applied uniformly with appropriate spreadability and show viscosity such that the agents do not sag.

The emulsion compositions for hair treating agents may be suitably used as hair coloring agents or hair perming agents as described above.

### EXAMPLES

The hair treating emulsion compositions according to the present invention will be described in Examples hereinbelow without limiting the scope of the invention. In Examples below, % is in terms of weight.

### [Evaluation Methods and Criteria]

Expert panelists (beauticians) evaluated the following items based on the following criteria using wigs (manufactured by Beaulax) that were certified for use in the beautician skill test.

### <Applicability>

The agents were applied to the wigs with a brush and the applicability was evaluated.

### Evaluation Criteria

AA: The agent was spread very smoothly and stayed on the hair very stably.
BB: The agent was spread smoothly and stayed on the hair stably.
CC: The agent showed bad spreadability and/or bad stability on the hair.
DD: The agent showed very bad spreadability and/or very bad stability on the hair.
   <Drying Degree of Agents after Standing>

The agents were applied to the wigs and were allowed to stand for 20 minutes. The drying degree of the agents was evaluated and the hair was combed through to evaluate the comb-catching degree.

### Evaluation Criteria

AA: The agent was substantially unchanged from after immediately applied, and the hair was combed through without catching the comb.
BB: The agent was slightly dried compared to immediately after application, but the hair was combed through without catching the comb.
CC: The agent was dried compared to immediately after application, and the hair slightly caught the comb when it was combed through.
DD: The agent was completely dried, and the hair could not be combed through.

### <Rinsability>

The agents were applied to the wigs and were allowed to stand for 30 minutes. The wigs were then rinsed with hot water approximately at 40° C. The rinsability of the agents was evaluated.

### Evaluation Criteria

AA: The agent was rinsed very quickly.
BB: The agent was rinsed quickly.
CC: The agent was greasy and was hard to rinse off.
DD: The agent was greasy and was very hard to rinse off.

### <Finger-Combing Properties in Hair Rinsing>

After the agents were rinsed off, the wigs were rinsed with hot water at approximately 40° C. and finger-combing properties were evaluated.

### Evaluation Criteria

AA: Very good
BB: Good
CC: Finger-combing properties were slightly low.
DD: Finger-combing properties were very low and the fingers were caught by the hair.

### <Finish Smoothness>

The wigs were then shampooed and conditioned with commercially available products, and were completely dried with a dryer. The finish smoothness was evaluated by touching the hair.

### Evaluation Criteria

AA: Very smooth
BB: Smooth
CC: No smoothness was felt.
DD: The hair was squeaky.

### <Finish Luster>

The wigs that had been shampooed and conditioned with commercially available products, and been completely dried with a dryer, were evaluated in terms of luster.

### Evaluation Criteria

AA: The hair was very lustrous.
BB: The hair was lustrous.
CC: The hair was not lustrous.
DD: The hair was not lustrous and looked dry.

### [Hair Coloring Agents]

Emulsion compositions for hair treating agents were prepared using the components in the amounts set forth in Tables 1 to 4. The emulsion compositions were used as hair coloring first agents. The first agent was mixed with a usual hair coloring second agent according to Table 5, in 1:1 (weight ratio) to give a hair coloring agent. The hair coloring agents were evaluated by the aforementioned methods under the foregoing criteria. The results are shown in Tables 1 to 4.

TABLE 1
<Hair coloring first agent>

| | Ex. 1 | Ex. 2 | Ex. 3 | Ex. 4 | Ex. 5 | Ex. 6 | Ex. 7 |
|---|---|---|---|---|---|---|---|
| Cetostearyl alcohol | 9 | 9 | 9 | 9 | 9 | 9 | 9 |
| Paraffin | 5 | 5 | 5 | 5 | 5 | 5 | 5 |
| Polyoxyethylene cetyl ether | 7 | 7 | 7 | 7 | 7 | 7 | 7 |
| Cetostearyltrimethylammonium | 2 | 2 | 2 | 2 | 2 | 2 | 2 |
| Glycerol | 14 | 18 | 7 | 23 | 3.2 | 28 | 1.2 |
| PEG-9M | 0.1 | 0.1 | 0.1 | 0.1 | 0.1 | 0.1 | 0.1 |
| Lanolin fatty acid | 5 | 5 | 5 | 5 | 5 | 5 | 5 |
| octyldodecyl | | | | | | | |
| Dimethyldiallylammonium | 1.5 | 1.5 | 1.5 | 1.5 | 1.5 | 1.5 | 1.5 |
| chloride/acrylamide/acrylic | | | | | | | |
| acid copolymer | | | | | | | |
| 28% Ammonia water | 5 | 5 | 5 | 5 | 5 | 5 | 5 |
| Sodium sulfite | 0.5 | 0.5 | 0.5 | 0.5 | 0.5 | 0.5 | 0.5 |
| Paraphenylenediamine | 0.1 | 0.1 | 0.1 | 0.1 | 0.1 | 0.1 | 0.1 |
| Resorcin | 0.08 | 0.08 | 0.08 | 0.08 | 0.08 | 0.08 | 0.08 |
| Meta-aminophenol | 0.05 | 0.05 | 0.05 | 0.05 | 0.05 | 0.05 | 0.05 |
| Purified water | Bal.* | Bal.* | Bal.* | Bal.* | Bal.* | Bal.* | Bal.* |
| Total amount | 100 | 100 | 100 | 100 | 100 | 100 | 100 |
| <Evaluation results> | | | | | | | |
| Applicability | AA | AA | AA | AA | BB | BB | BB |
| Drying degree during standing | AA | AA | AA | AA | BB | AA | BB |
| Rinsability | AA | AA | AA | AA | AA | BB | AA |
| Finger-combing properties in | AA | AA | AA | BB | AA | BB | AA |
| hair rinsing | | | | | | | |
| Finish smoothness | AA | AA | AA | AA | AA | BB | AA |
| Finish luster | AA | AA | AA | BB | AA | BB | BB |

| | | | Ex. | Ex. | Ex. | Ex. |
|---|---|---|---|---|---|---|
| | Ex. 8 | Ex. 9 | 10 | 11 | 12 | 13 |
| Cetostearyl alcohol | 9 | 9 | 9 | 9 | 9 | 9 |
| Paraffin | 5 | 5 | 5 | 5 | 5 | 5 |
| Polyoxyethylene cetyl ether | 7 | 7 | 7 | 7 | 7 | 7 |
| Cetostearyltrimethylammonium | 2 | 2 | 2 | 2 | 2 | 2 |
| Glycerol | 14 | 14 | 14 | 14 | 14 | 14 |
| PEG-9M | 0.5 | 0.06 | 0.9 | 0.04 | 1.8 | 0.02 |
| Lanolin fatty acid | 5 | 5 | 5 | 5 | 5 | 5 |
| octyldodecyl | | | | | | |
| Dimethyldiallylammonium | 1.5 | 1.5 | 1.5 | 1.5 | 1.5 | 1.5 |
| chloride/acrylamide/acrylic | | | | | | |
| acid copolymer | | | | | | |
| 28% Ammonia water | 5 | 5 | 5 | 5 | 5 | 5 |
| Sodium sulfite | 0.5 | 0.5 | 0.5 | 0.5 | 0.5 | 0.5 |
| Paraphenylenediamine | 0.1 | 0.1 | 0.1 | 0.1 | 0.1 | 0.1 |
| Resorcin | 0.08 | 0.08 | 0.08 | 0.08 | 0.08 | 0.08 |
| Meta-aminophenol | 0.05 | 0.05 | 0.05 | 0.05 | 0.05 | 0.05 |
| Purified water | Bal.* | Bal.* | Bal.* | Bal.* | Bal.* | Bal.* |
| Total amount | 100 | 100 | 100 | 100 | 100 | 100 |
| <Evaluation results> | | | | | | |
| Applicability | AA | AA | AA | BB | BB | BB |
| Drying degree during standing | AA | AA | AA | BB | AA | BB |
| Rinsability | AA | AA | AA | AA | AA | BB |
| Finger-combing properties in | AA | AA | AA | AA | BB | AA |
| hair rinsing | | | | | | |
| Finish smoothness | AA | AA | AA | AA | AA | BB |
| Finish luster | AA | AA | AA | AA | AA | BB |

*Bal: Balance
The values in the table indicate wt % relative to the total (100) of the components.

TABLE 2
<Hair coloring first agent>

| | | Ex. | Ex. | Ex. | Ex. | Ex. | Ex. | Ex. | Ex. | Ex. | Ex. | Ex. |
|---|---|---|---|---|---|---|---|---|---|---|---|---|
| | Ex. 14 | 15 | 16 | 17 | 18 | 19 | 20 | 21 | 22 | 23 | 24 | 25 |
| Cetostearyl alcohol | 9 | 9 | 9 | 9 | 9 | 9 | 9 | 9 | 9 | 9 | 9 | 9 |
| Paraffin | 5 | 5 | 5 | 5 | 5 | 5 | 5 | 5 | 5 | 5 | 5 | 5 |
| Polyoxyethylene cetyl ether | 7 | 7 | 7 | 7 | 7 | 7 | 7 | 7 | 7 | 7 | 7 | 7 |
| Cetostearyltrimethylammonium | 2 | 2 | 2 | 2 | 2 | 2 | 2 | 2 | 2 | 2 | 2 | 2 |
| Glycerol | 14 | 14 | 14 | 14 | 14 | 14 | 14 | 14 | 14 | 14 | 14 | 14 |
| PEG-9M | 0.1 | 0.1 | 0.1 | 0.1 | 0.1 | 0.1 | 0.1 | 0.1 | 0.1 | 0.1 | 0.1 | 0.1 |
| Lanolin fatty acid | 9 | 0.6 | 13 | 0.25 | 18 | 0.12 | 5 | 5 | 5 | 5 | 5 | 5 |
| octyldodecyl | | | | | | | | | | | | |
| Dimethyldiallylammonium | 1.5 | 1.5 | 1.5 | 1.5 | 1.5 | 1.5 | 2.8 | 0.5 | 3.8 | 0.25 | 5.8 | 0.12 |
| chloride/acrylamide/acrylic | | | | | | | | | | | | |
| acid copolymer | | | | | | | | | | | | |
| 28% Ammonia water | 5 | 5 | 5 | 5 | 5 | 5 | 5 | 5 | 5 | 5 | 5 | 5 |
| Sodium sulfite | 0.5 | 0.5 | 0.5 | 0.5 | 0.5 | 0.5 | 0.5 | 0.5 | 0.5 | 0.5 | 0.5 | 0.5 |
| Paraphenylenediamine | 0.1 | 0.1 | 0.1 | 0.1 | 0.1 | 0.1 | 0.1 | 0.1 | 0.1 | 0.1 | 0.1 | 0.1 |
| Resorcin | 0.08 | 0.08 | 0.08 | 0.08 | 0.08 | 0.08 | 0.08 | 0.08 | 0.08 | 0.08 | 0.08 | 0.08 |
| Meta-aminophenol | 0.05 | 0.05 | 0.05 | 0.05 | 0.05 | 0.05 | 0.05 | 0.05 | 0.05 | 0.05 | 0.05 | 0.05 |
| Purified water | Bal.* | Bal.* | Bal.* | Bal.* | Bal.* | Bal.* | Bal.* | Bal.* | Bal.* | Bal.* | Bal.* | Bal.* |
| Total amount | 100 | 100 | 100 | 100 | 100 | 100 | 100 | 100 | 100 | 100 | 100 | 100 |
| <Evaluation results> | | | | | | | | | | | | |
| Applicability | AA | AA | AA | AA | AA | AA | AA | AA | BB | BB | BB | BB |
| Drying degree during standing | AA | AA | AA | AA | AA | BB | AA | AA | AA | AA | AA | BB |
| Rinsability | AA | AA | AA | AA | BB | AA | AA | AA | AA | AA | BB | AA |
| Finger-combing properties in | AA | AA | BB | AA | BB | AA | AA | AA | BB | AA | BB | AA |
| hair rinsing | | | | | | | | | | | | |
| Finish smoothness | AA | AA | AA | BB | AA | BB | AA | AA | AA | AA | AA | AA |
| Finish luster | AA | AA | AA | BB | BB | BB | AA | AA | AA | AA | AA | AA |

*Bal: Balance
The values in the table indicate wt % relative to the total (100) of the components.

TABLE 3
<Hair coloring first agent>

| | | Ex. | Ex. | Comp. | Comp. | Comp. | Comp. | Comp. | Comp. | Comp. | Comp. | Comp. |
|---|---|---|---|---|---|---|---|---|---|---|---|---|
| | Ex. 26 | 27 | 28 | Ex. 1 | Ex. 2 | Ex. 3 | Ex. 4 | Ex. 5 | Ex. 6 | Ex. 7 | Ex. 8 | Ex. 9 |
| Cetostearyl alcohol | 9 | 9 | 9 | 9 | 9 | 9 | 9 | 9 | 9 | 9 | 9 | 9 |
| Paraffin | 5 | 5 | 5 | 5 | 5 | 5 | 5 | 5 | 5 | 5 | 5 | 5 |
| Polyoxyethylene cetyl ether | 7 | 7 | 7 | 7 | 7 | 7 | 7 | 7 | 7 | 7 | 7 | 7 |
| Cetostearyltrimethylammonium | 2 | 2 | 2 | 2 | 2 | 2 | 2 | 2 | 2 | 2 | 2 | 2 |
| Glycerol | 14 | 14 | 14 | 32 | 0.8 | 0 | 14 | 14 | 14 | 14 | 14 | 14 |
| PEG-9M | 0.1 | 0.1 | 0.1 | 0.1 | 0.1 | 0.1 | 2.2 | 0.008 | 0 | 0.1 | 0.1 | 0.1 |
| Lanolin fatty acid | 5 | 5 | 5 | 5 | 5 | 5 | 5 | 5 | 5 | 22 | 0.08 | 0 |
| octyldodecyl | | | | | | | | | | | | |
| Dimethyldiallylammonium | 6.2 | 0.08 | 0 | 1.5 | 1.5 | 1.5 | 1.5 | 1.5 | 1.5 | 1.5 | 1.5 | 1.5 |
| chloride/acrylamide/acrylic | | | | | | | | | | | | |
| acid copolymer | | | | | | | | | | | | |
| 28% Ammonia water | 5 | 5 | 5 | 5 | 5 | 5 | 5 | 5 | 5 | 5 | 5 | 5 |
| Sodium sulfite | 0.5 | 0.5 | 0.5 | 0.5 | 0.5 | 0.5 | 0.5 | 0.5 | 0.5 | 0.5 | 0.5 | 0.5 |
| Paraphenylenediamine | 0.1 | 0.1 | 0.1 | 0.1 | 0.1 | 0.1 | 0.1 | 0.1 | 0.1 | 0.1 | 0.1 | 0.1 |
| Resorcin | 0.08 | 0.08 | 0.08 | 0.08 | 0.08 | 0.08 | 0.08 | 0.08 | 0.08 | 0.08 | 0.08 | 0.08 |
| Meta-aminophenol | 0.05 | 0.05 | 0.05 | 0.05 | 0.05 | 0.05 | 0.05 | 0.05 | 0.05 | 0.05 | 0.05 | 0.05 |
| Purified water | Bal.* | Bal.* | Bal.* | Bal.* | Bal.* | Bal.* | Bal.* | Bal.* | Bal.* | Bal.* | Bal.* | Bal.* |
| Total amount | 100 | 100 | 100 | 100 | 100 | 100 | 100 | 100 | 100 | 100 | 100 | 100 |
| <Evaluation results> | | | | | | | | | | | | |
| Applicability | BB | BB | BB | BB | CC | DD | DD | CC | DD | BB | AA | AA |
| Drying degree during standing | AA | BB | BB | AA | DD | DD | AA | DD | DD | AA | CC | DD |
| Rinsability | AA | AA | BB | CC | AA | AA | BB | CC | CC | CC | AA | AA |
| Finger-combing properties in | AA | AA | AA | DD | AA | AA | BB | AA | AA | DD | BB | CC |
| hair rinsing | | | | | | | | | | | | |
| Finish smoothness | AA | AA | AA | CC | AA | AA | AA | BB | CC | AA | DD | DD |
| Finish luster | AA | AA | AA | BB | CC | DD | BB | BB | BB | BB | CC | DD |

*Bal: Balance
The values in the table indicate wt % relative to the total (100) of the components.

TABLE 4
<Hair coloring first agent>

| | Ex. 29 | Ex. 30 | Ex. 31 | Ex. 32 | Ex. 33 | Ex. 34 |
|---|---|---|---|---|---|---|
| Cetostearyl alcohol | 9 | 9 | 9 | 9 | 9 | 9 |
| Paraffin | 5 | 5 | 5 | 5 | 5 | 5 |
| Polyoxyethylene cetyl ether | 7 | 7 | 7 | 7 | 7 | 7 |
| Cetostearyltrimethylammonium | 2 | 2 | 2 | 2 | 2 | 2 |
| Glycerol | | | 7 | 7 | | |
| Diglycerol | 14 | | 7 | | 7 | 4 |
| Dipropyleneglycol | | 14 | | 7 | 7 | 4 |
| PEG-9M | 0.1 | 0.1 | 0.1. | 0.1 | 0.1 | 0.1 |
| Lanolin fatty acid | 5 | 5 | 5 | 5 | 5 | 5 |
| octyldodecyl | | | | | | |
| Dimethyldiallylammonium | 1.5 | 1.5 | 1.5 | 1.5 | 1.5 | 1.5 |
| chloride/acrylamide/acrylic | | | | | | |
| acid copolymer | | | | | | |
| 28% Ammonia water | 5 | 5 | 5 | 5 | 5 | 5 |
| Sodium sulfite | 0.5 | 0.5 | 0.5 | 0.5 | 0.5 | 0.5 |
| Paraphenylenediamine | 0.1 | 0.1 | 0.1 | 0.1 | 0.1 | 0.1 |
| Resorcin | 0.08 | 0.08 | 0.08 | 0.08 | 0.08 | 0.08 |
| Meta-aminophenol | 0.05 | 0.05 | 0.05 | 0.05 | 0.05 | 0.05 |
| Purified water | Bal.* | Bal.* | Bal.* | Bal.* | Bal.* | Bal.* |
| Total amount | 100 | 100 | 100 | 100 | 100 | 100 |
| Applicability | AA | AA | AA | AA | AA | AA |
| Drying degree during standing | AA | AA | AA | AA | AA | AA |
| Rinsability | AA | AA | AA | AA | AA | AA |
| Finger-combing properties in | AA | AA | AA | AA | AA | AA |
| hair rinsing | | | | | | |
| Finish smoothness | AA | AA | AA | AA | AA | AA |
| Finish luster | AA | AA | AA | AA | AA | AA |

*Bal: Balance
The values in the table indicate wt % relative to the total (100) of the components.

TABLE 5
<Usual hair coloring second agent>

| | Usual second agent |
|---|---|
| Cetanol | 4 |
| Paraffin | 1 |
| Polyoxyethylene cetyl ether | 1.5 |
| Cetostearyltrimethylammonium | 0.5 |
| Phenacetin | 0.1 |
| Tetrasodium hydroxyethanediphosphonate | 0.05 |
| Phosphoric acid | 0.05 |
| Hydrogen peroxide | 6 |
| Purified water | Balance |
| Total amount | 100 |

The values in the table indicate wt % relative to the total (100) of the components.

### [Hair Perming Agents]

Hair treating emulsion compositions according to Tables 6 and 7 were used as a hair perming first agent (preparation A-1) and/or second agent (preparation A-2), together with a usual hair perming first agent (preparation B-1) or second agent (preparation B-2) according to Tables 6 and 7, as a hair perming agent in combination.

These agents were used as hair perming agents in various combinations and were evaluated by the above-described methods under the above-described criteria. The first agent and second agent were used in 1:1 (weight ratio). The results are shown in Table 8.

TABLE 6
<Hair perming first agent>

| | Preparation | Preparation |
|---|---|---|
| | A-1 | B-1 |
| Cetostearyl alcohol | 9 | 9 |
| Paraffin | 5 | 5 |
| Polyoxyethylene cetyl ether | 7 | 7 |
| Cetostearyltrimethylammonium | 2 | 2 |
| Glycerol | 14 | 0 |
| PEG-9M | 0.1 | 0 |
| Lanolin fatty acid octyldodecyl | 5 | 0 |
| Dimethyldiallylammonium | 1.5 | 0 |
| chloride/acrylamide/acrylic acid copolymer | | |
| 28% Ammonia water | 3 | 3 |
| Ammonium thioglycolate | 15 | 15 |
| Purified water | Balance | Balance |
| Total amount | 100 | 100 |

The values in the table indicate wt % relative to the total (100) of the components.

TABLE 7
<Hair perming second agent>

| | Preparation | Preparation |
|---|---|---|
| | A-2 | B-2 |
| Cetostearyl alcohol | 9 | 9 |
| Paraffin | 5 | 5 |
| Polyoxyethylene cetyl ether | 7 | 7 |
| Cetostearyltrimethylammonium | 2 | 2 |
| Glycerol | 14 | 0 |
| PEG-9M | 0.1 | 0 |
| Lanolin fatty acid octyldodecyl | 5 | 0 |
| Dimethyldiallylammonium | 1.5 | 0 |
| chloride/acrylamide/acrylic acid copolymer | | |
| Phosphoric acid | 0.25 | 0.25 |
| Sodium hydroxide | 0.12 | 0.12 |
| Sodium bromate | 7 | 7 |
| Purified water | Balance | Balance |
| Total amount | 100 | 100 |

The values in the table indicate wt % relative to the total (100) of the components.

TABLE 8
<Perming> Evaluation results

| | Ex. 35 | Ex. 36 | Ex. 37 | Comp. Ex. 10 |
|---|---|---|---|---|
| Perming first agent | Preparation | Preparation | Preparation | Preparation |
| | A-1 | A-1 | B-1 | B-1 |
| Perming second agent | Preparation | Preparation | Preparation | Preparation |
| | A-2 | B-2 | A-2 | B-2 |
| Applicability of first agent | AA | AA | DD | DD |
| Applicability of second agent | AA | DD | AA | DD |
| Drying degree of first agent | AA | AA | DD | DD |
| during standing | | | | |
| Drying degree of second agent | AA | DD | AA | DD |
| during standing | | | | |
| Rinsability of first agent | AA | AA | DD | DD |
| Rinsability of second agent | AA | DD | AA | DD |
| Finger-combing properties in | AA | AA | DD | DD |
| rinsing first agent | | | | |
| Finger-combing properties in | AA | DD | AA | DD |
| rinsing second agent | | | | |
| Finish smoothness | AA | BB | BB | DD |
| Finish luster | AA | BB | BB | DD |

1. An emulsion composition for hair treating agents, which composition comprises a lubricant, a surfactant and water and further comprises:
0.5 to 15 wt % of at least one polyhydric alcohol selected from the group consisting of glycerol, diglycerol and dipropylene glycol;
0.005 to 1 wt % of a highly polymerized polyethylene glycol; and
0.05 to 10 wt % of lanolin and/or a lanolin derivative.

2. The emulsion composition for hair treating agents according to claim 1, which further comprises 0.05 to 3 wt % of an amphoteric polymer.

3. The emulsion composition for hair treating agents according to claim 2, wherein the amphoteric polymer is a dimethyldiallylammonium chloride/acrylamide/acrylic acid copolymer.

4. The emulsion composition for hair treating agents according to claim 1, wherein the highly polymerized polyethylene glycol has a viscosity average molecular weight in the range of 20,000 to 4,500,000.

5. The emulsion composition for hair treating agents according to claim 1, wherein the lanolin and/or lanolin derivative is a lanolin fatty acid octyldodecyl.

6. The emulsion composition for hair treating agents according to claim 1, wherein the emulsion composition is used as a hair coloring agent.

7. The emulsion composition for hair treating agents according to claim 1, wherein the emulsion composition is used as a hair perming agent.

8. The emulsion composition for hair treating agents according to claim 2, wherein the highly polymerized polyethylene glycol has a viscosity average molecular weight in the range of 20,000 to 4,500,000.

9. The emulsion composition for hair treating agents according to claim 3, wherein the highly polymerized polyethylene glycol has a viscosity average molecular weight in the range of 20,000 to 4,500,000.

10. The emulsion composition for hair treating agents according to claim 2, wherein the lanolin and/or lanolin derivative is a lanolin fatty acid octyldodecyl.

11. The emulsion composition for hair treating agents according to claim 3, wherein the lanolin and/or lanolin derivative is a lanolin fatty acid octyldodecyl.

12. The emulsion composition for hair treating agents according to claim 4, wherein the lanolin and/or lanolin derivative is a lanolin fatty acid octyldodecyl.

13. The emulsion composition for hair treating agents according to claim 2, wherein the emulsion composition is used as a hair coloring agent.

14. The emulsion composition for hair treating agents according to claim 3, wherein the emulsion composition is used as a hair coloring agent.

15. The emulsion composition for hair treating agents according to claim 4, wherein the emulsion composition is used as a hair coloring agent.

16. The emulsion composition for hair treating agents according to claim 5, wherein the emulsion composition is used as a hair coloring agent.

17. The emulsion composition for hair treating agents according to claim 2, wherein the emulsion composition is used as a hair perming agent.

18. The emulsion composition for hair treating agents according to claim 3, wherein the emulsion composition is used as a hair penning agent.

19. The emulsion composition for hair treating agents according to claim 4, wherein the emulsion composition is used as a hair penning agent.

20. The emulsion composition for hair treating agents according to claim 5, wherein the emulsion composition is used as a hair perming agent.
